(19) United States
(12) Patent Application Publication
(10) Pub. No.: US 2026/0252745 A1
(43) Pub. Date: Aug. 27, 2026

(54) METHOD AND COMPUTER PROGRAMME FOR CREATING A DIGITAL 3D MODEL, AND METHOD FOR ADJUSTING OR PRODUCING AN ORTHOPAEDIC DEVICE

(71) Applicant: Ottobock SE & Co. KGaA, Duderstadt (DE)

(72) Inventor: Marvin Mouroum, Duderstadt (DE)

(21) Appl. No.: 18/994,421

(22) PCT Filed: Jul. 11, 2023
(86) PCT No.: PCT/EP2023/069184

§ 371 Date: Jan. 14, 2025

(30) Foreign Application Priority Data
Jul. 15, 2022 (DE) 10 2022 117 757.2

(51) Int. Cl. G06F 30/10; G06V 10/10; G06V 10/82; G06V 40/10

(52) U.S. Cl. CPC G06F 30/10; G06V 10/17; G06V 10/82; G06V 40/10

(57) ABSTRACT

The invention relates to a method for creating a digital 3D model of a patient's body part, comprising at least one first body part section that is adjacent to at least one articulated second body part section, such that the first body part section can be moved by means of the articulated second body part section, wherein the method comprises the following steps performed by a digital processing device:
providing digital, camera-based image data that contains the part of the patient's body to be digitally modelled from different imaging directions,
identifying the at least one first body part section from the digital image data provided and creating a first 3D body part section model for the identified first body part section based on the digital image data provided and depth information provided on the identified first body part section,
identifying the at least one articulated second body part section from the digital image data provided, and
creating the digital 3D model of the body part to be modelled depending on the first 3D body part section model created and the identified articulated second body part section.

The invention relates to a method for creating a digital 3D model of a patient's body part, comprising at least one first body part section and at least one articulated second body part section that is adjacent to the first body part section, such that the first body part section can be moved by means of the articulated second body part section. The invention also relates to an associated computer program.

Furthermore, the invention relates to a method for adjusting or producing an orthopedic device.

Within the context of the present invention, orthopedic devices refer, in particular, to orthoses, prostheses, exoskeletons and possibly wheelchairs. Orthoses are products that brace, support, protect or restrict the freedom of movement of a body part of the patient in order to prevent excessive strain. Conversely, prostheses replace body parts that are missing or are no longer there. Specifically, exoskeletons are mechanical support structures that serve to brace, support or protect the patient's main musculoskeletal system.

In the following, the term patient refers to any user of the orthopedic device. It is therefore the person wearing the orthopedic device.

Each orthopedic device is arranged on a body part of the patient. It does not necessarily have to come into contact with the patient's skin. For example, orthoses and exoskeletons are often worn over clothing, so that said clothing, for example trousers, is located between the orthopedic device and the patient's skin. Nevertheless, a knee orthosis, for example, is attached to the patient's knee or leg. A prosthesis always has an interface element connected to an amputation stump or another body part, said element being attached to the respective body part. In the case of a leg prosthesis, for example, a prosthesis socket is used that acts as the interface between the prosthesis and the residual limb. In this case, the residual limb would be the patient's body part. A liner is usually used between the skin surface of the patient's residual limb and the prosthesis socket in order to reduce the shear forces acting on the skin.

A prosthesis socket for a residual limb is generally produced from a rigid (barely deformable) material, such as a fiber-reinforced plastic, and forms an important part of the interface between the residual limb and the prosthesis socket arranged on the prosthesis socket. Prosthetic sockets have been used for many years, particularly for leg prostheses that are to be attached to a residual limb, such as a femoral stump.

In particular, prosthetic sockets for leg amputees are exposed to particular stresses in daily use. When walking, the patient's full weight is on the prosthesis socket and thus particularly on the residual limb arranged in the prosthesis socket. It is therefore very important to adjust the prosthesis socket as optimally as possible to the patient's individual conditions and needs, in particular to the shape and geometry of the respective body part.

To be able to optimally adjust and set an orthopedic device to the patient's conditions, particularly in connection with a digital data processing system, it is necessary to first create as precise a model as possible of the body part that at least partially comes into contact with the orthopedic device and is to be treated with said device.

In practice, it is known to use a scanning device to scan the body part under consideration and create a digital 3D model of the modelling body part. For example, camera-based image data of the body part can be captured and a model of the body part subsequently created in the digital data processing system on the basis of this image data. The orthopedic device can be configured, for example, or a physical model created on the basis of the 3D model.

If the patient remains sufficiently still during the scanning process, the camera-based capturing and creation of the 3D model is usually uncomplicated. However, patients are often unable to keep the affected body part sufficiently still for a scan to be completed with a good result. This applies in particular to cases in which the body part is to be measured without a load and without a holding device. It can also be difficult or even impossible for children or patients with a spasticity to keep the body part sufficiently still.

For example, if an upper or lower leg is measured, bending the knee joint between the upper and lower leg can cause the scanning procedure to be aborted, as the entire body part, formed of the lower leg, knee and upper leg, is considerably deformed. The reconstruction algorithm is then no longer able to assign the individual pixels accordingly and must therefore be cancelled.

The present invention therefore aims to provide an improved method for creating a digital 3D model of a body part that is to be treated with an orthopedic device.

According to the invention, the task is solved using the method in accordance with claim 1. Advantageous embodiments of the invention are then to be found in the corresponding sub-claims.

According to claim 1, a method for creating a digital 3D model of a patient's body part is proposed, the body part having at least one first body part section that is adjacent to at least one articulated second body part section, such that the first body part section can be moved by means of the articulated second body part section. More than one first body part section, which is rigid in particular, can be arranged on the articulated second body part section.

The method comprises the following steps, carried out by a digital data processing device:
providing digital, camera-based image data that contains the part of the patient's body to be digitally modelled from different imaging directions,
identifying the at least one first body part section from the digital image data provided and creating a first 3D body part section model for the identified first body part section based on the digital image data provided and depth information provided on the identified first body part section,
identifying the at least one articulated second body part section from the digital image data provided, and
creating the digital 3D model of the body part to be modelled depending on the first 3D body part section model created and the identified articulated second body part section.

According to the invention, it is proposed that the individual body part sections are identified separately and the body part section model is then created at least for the first body part section on the basis of the separate identification of the body part sections, so that the 3D model of the body part to be modelled can then be created on the basis thereof. The body part to be modelled is divided into individual body part sections, as these individual body part sections in and of themselves only change shape slightly. The body part sections identified in this manner are later reconstructed and assembled, wherein the articulated body part section does not necessarily have to form part of the model. In this case, each rigid body part section can be individually reconstructed, regardless of the joint bending, and subsequently assembled, with reference to the joint, to produce a complete model.

The present invention thus renders it possible to create a 3D model even if the patient moves, which is especially advantageous when scanning children, as they rarely stay still for a long time.

The depth information can be determined from the digital image data provided, for example, if it contains the body part from different imaging directions. However, it is also conceivable that this depth information originates from a different imaging source, for example it is directly recorded by means of a laser scanner, preferably in addition to the camera data.

According to one embodiment, it is provided for that a second 3D body part section model for the identified articulated second body part section is created based on the digital image data provided and depth information provided on the identified articulated second body part section, wherein the digital 3D model of the body part to be modelled is created depending on the first 3D body part section model created and the second 3D body part section model created.

Advantageously, a body part section model is also created for the articulated second body part section, which has been identified beforehand, so that a body part section model is created for both the first body part section or sections and the articulated second body part section, the digital 3D model subsequently being created on the basis of these body part section models. In this embodiment, the articulated body part section constitutes part of the 3D model.

According to one embodiment, it is provided that at least two body part sections are further identified as a function of a relationship to one another.

Knowing the relationships between the individual body part sections, the individual body part sections can be identified from the digital image data and at least one body part section model can then be created.

According to one embodiment, it is intended that the articulated second body part section is identified as a function of a range of motion of the joint or that the first body part section is identified as a function of a range of motion of a joint adjacent to the first body part section or of the identified articulated second body part section.

Knowing the range of motion of the joint, it is possible to identify the first body part section arranged on the joint, wherein said sections may be rigid in particular, in order to create the body part section model for the respective identified body part sections. Knowing the range of motion of the joint, a possible movement of the adjacent first body part section can thus be predicted, thereby improving identification. However, the joint itself can also be better identified when the range of motion is known. This can also improve reconstruction, as possible deviations from the initial position can be predicted at the start of the scan. Deviations that can no longer be explained by the range of motion of the joint, on the other hand, must be artefacts that can be discarded.

According to one embodiment, it is provided that the body part sections of the body part to be modelled are captured in a joint scanning procedure, wherein the at least one body part section model is created separately.

The entire body part to be modelled in a 3D model is recorded in a joint scanning procedure, the individual body part sections however being identified and respective body part section models being created separately. To create the 3D model, the individual body part section models, which have been created separately, are then joined to produce an overall model in order to reconstruct the body part to be modelled.

According to one embodiment, it is intended that a machine learning system is provided which has learned a correlation of digital image data as input data with the respective identification of the corresponding body part section as output data, wherein the image data provided is entered into the machine learning system as input data for the purpose of identification and an identification of the corresponding body part section is obtained as output data.

The machine learning system, which may be an artificial neural network in one preferred embodiment, gets the digital image data as input data, wherein the identification of one of the body part sections is obtained as an output. For example, the identification can be performed by placing a so-called bounding box around the identified body part section, said bounding box corresponding to the outlines of the respective body part sections. However, it is also conceivable that the body part sections to be identified are given a particular color in the digital image data, wherein image data is available as an output of the machine learning system, said image data containing the identified body part section in a predetermined color. Finally, by entering the digital image data as input data, the machine learning system determines an identifier for identifying the corresponding body part section as output data, whereby this identifier contains the identification of the body part section within the image data.

Here, a training data set for training the machine learning system contains digital image data to which one identifier is allocated in each case for identifying the respective body part section. This means that a corresponding identification of the body part section to be learned is allocated to each set of image data. To train the machine learning system, a range of training data sets is provided in order to condition the machine learning system accordingly.

According to one embodiment, it is provided that the digital, camera-based image data is continuously provided to the data processing device.

In one preferred embodiment, continuously providing the image data means that the 3D model of the body part can also be continuously created.

According to one preferred embodiment, it is intended that, by means of a mobile image sensor, the digital, camera-based image data of the patient's body part to be modelled is recorded and supplied to the digital data processing device in order to create the 3D body part model. For example, such a mobile image sensor can be a hand-operated image sensor.

According to the invention, the task is also solved by way of the computer program according to claim 12, the computer program having program code means that are configured to perform the method described above when the computer program is run a data processing system.

According to the invention, the task is also solved by way of the method for creating or producing an orthopedic device according to claim 13, comprising the steps:
creating a 3D body part model of the body part to be equipped with the orthopedic device according to the method described above, and
adjusting or producing the orthopedic device using the 3D body part model.

The invention is explained in more detail by means of the attached figures. They show:

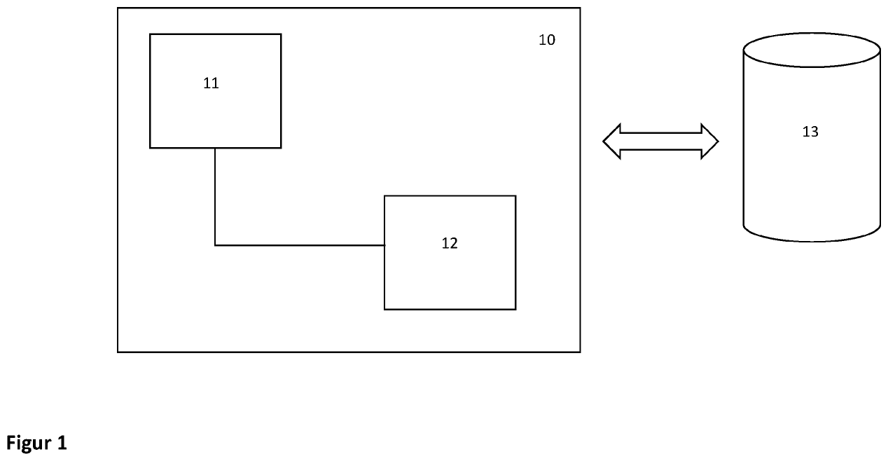

FIG. 1 schematic representation of the device according to the invention;

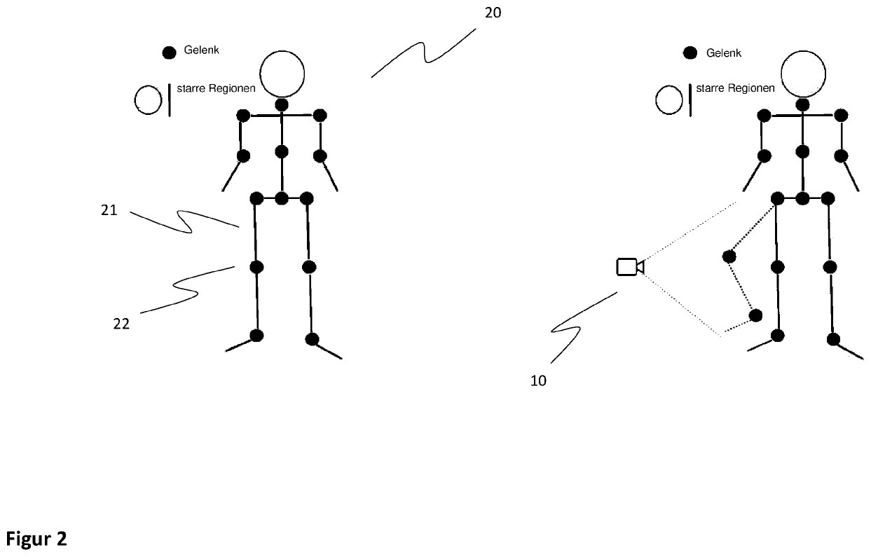

FIG. 2 schematic representation of a scanning procedure;

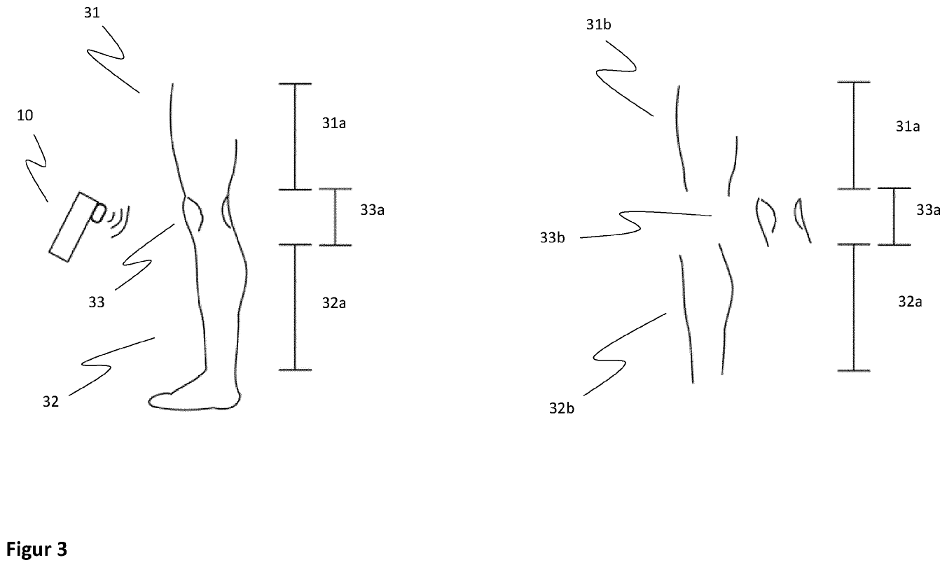

FIG. 3 schematic representation of a reconstruction after scanning;

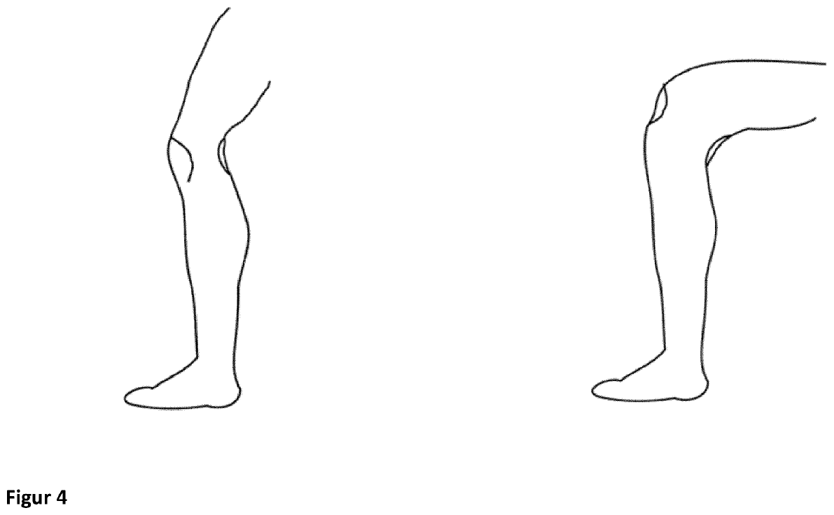

FIG. 4 illustrative representation of movements;

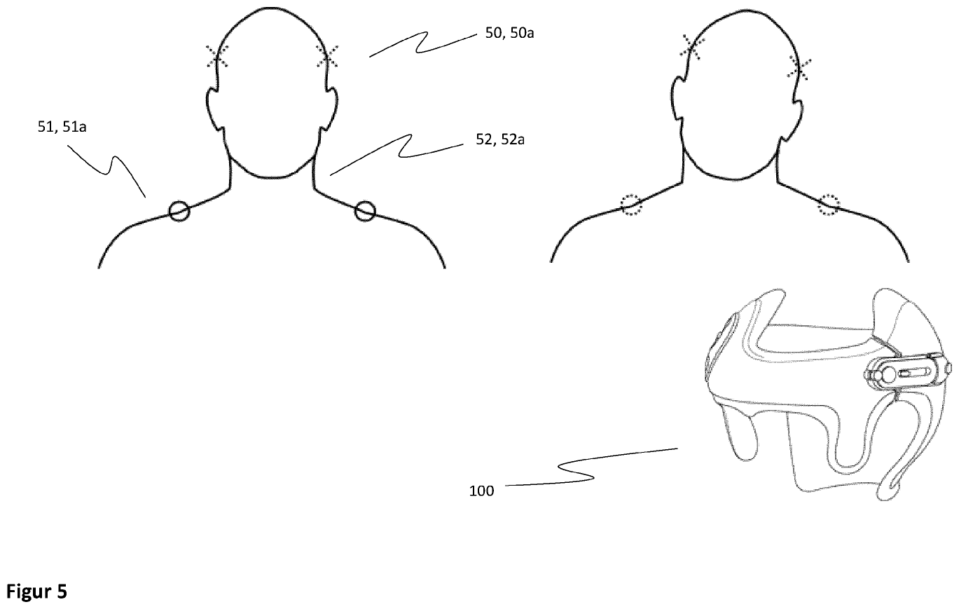

FIG. 5 representation of a further embodiment example.

In a highly simplified schematic representation, FIG. 1 shows a mobile terminal device 10 that has a scanning device 11 and a data processing device 12. With the aid of the scanning device 11, digital image data of the body part to be modelled can be recorded and transmitted to the data processing device in order to create the 3D model of the body part to be modelled. Such a 3D model which has been created can then be stored, for example, in a digital memory 13.

On the left-hand side of FIG. 2 is an abstract representation of a person 20 with rigid regions 21 as first body part sections and joints 22 as second body part sections. The abstract representation of the person 20 on the left-hand side depicts a starting position in which the person 20 does not exhibit any movement at all.

With the aid of a mobile terminal device 10, the person's 20 right upper and lower leg is now to be captured and modelled. The person 20 may move their upper and/or lower leg in the process, as both the upper leg is movably arranged on the hip joint and the lower leg can be moved at the knee joint in relation to the upper leg.

Such a movement, as shown on the right-hand side of FIG. 2, which occurs during a scanning procedure, would normally cause the procedure to be aborted as the image recognition is no longer able to allocate the sections of the body part that are moving relative to each other.

FIG. 3 shows a highly simplified schematic representation of such a scanning procedure. First, the body part 30 to be modelled is scanned using a mobile terminal device 10. The lower leg, upper leg and the knee joint between them are separately and individually identified with the aid of a machine learning system, which is run on the mobile terminal device 10, using the camera-based image data recorded during scanning. Irrespective of the bending of the knee, as shown in FIG. 4, the three body part sections are identified.

The upper leg 31 and the lower leg 32 constitute the first body part sections which are, in particular, rigid. In particular, this means that the upper leg 31 and the lower leg 32 do not have a further joint in order to change the basic shape. Of course, there may also be soft tissue present in rigid body part sections, the former tending to briefly change shape due to external forces.

The knee joint 33 between the upper leg 31 and the lower leg 32 means that the upper leg 31 and lower leg 32 can be moved relative to each other. In the embodiment example in FIG. 3, the knee joint 33 thus forms the articulated second body part section. Such a scanning procedure is depicted on the left-hand side of FIG. 3. The three body part sections 31a to 33a are identified in the process. On the basis of this identification of the three body part sections 31a to 33a in the digital image data, one body part section model 31b to 33b is subsequently created for each respective body part section 31a to 33a.

After the separate creation of the individual body part section models 31b to 33b, the desired 3D model can then be created on the basis thereof.

An orthopedic device can then be adjusted based on the 3D model created. Specifically, this comprises the creation of the orthopedic device based on the 3D model and the configuration of the orthopedic device based on the 3D model.

FIG. 5 depicts an embodiment example in which a head 50 is to be scanned in order to produce an orthopedic device 100. The first body part section 50a is rigid and does not have any joints. A further first body part section 51a, the shoulder area 51, is to be considered a rigid body area for the scanning procedure. Between the head 50 and the shoulder area 51 is the neck 52, which is a joint in the context of the present invention and can therefore be considered a second body part section 52a. The neck 52 connects the head 50 with the shoulder area 51 in such a way that the head 50 can perform a relative movement in relation to the shoulder area 51.

For example, if a child's head 50 is to be scanned, it is difficult to limit the scanning area to the head from the outset, as both shoulders and neck are usually scanned as well. However, since the child usually moves its head during the scanning procedure, the scan is aborted or results in artefacts. This can be avoided by recognizing the joint area, the neck 52, and limiting the reconstruction to the rigid section, i.e. the head 50.

In the embodiment example in FIG. 5, a head orthosis 100 is to be created which is used, for example, to treat plagiocephaly in babies. To this end, the head 50, or more specifically the upper area of the head 50, must be scanned. There are no joints there, meaning it is a rigid body part section. However, it can move as a whole via the neck joint 52, for example. In addition, it is almost impossible to only scan the head 50, as the scanner would normally capture the neck 52 and the shoulders 51 as well.

The points marked with an X in FIG. 5 in the upper head area are located in a rigid first body part section 50a. Their position in relation to each other remains the same, even when the head 50 moves. The two points marked with an O in the shoulder area do not change their position relative to each other either.

However, if the patient tilts their head, as shown on the right-hand side of FIG. 5, the position of the points marked with an X in the head area changes to those marked with an O in the shoulder area, which normally leads to artefacts and can cause the reconstruction or scanning process to be aborted.

According to the invention, the problem is solved in that the neck 52 is recognized as an articulated area and the head 50 and shoulder area 52 are each reconstructed independently from each other as rigid areas.

In the embodiment example in FIG. 5, the reconstruction of the head 50 is sufficient for the head orthosis 100, so that the shoulder area 52 can be discarded. A corresponding model is therefore only created for head 50.

### REFERENCE LIST

10 mobile hand device
11 scanning device
12 data processing device
13 digital memory
20 person
21 upper leg/first body part section
22 knee joint/second body part section
31 upper leg
32 lower leg
33 knee joint
31a first body part section as an upper leg
32a first body part section as a lower leg
33a second body part section as a knee joint
31b first body part section model as an upper leg
32b first body part section model as a lower leg
33b second body part section model as a knee joint
50 head
50a first body part section
51 shoulder area
51a first body part section
52 neck
52a second body part section
100 orthopedic device

1. A method for creating a digital 3D model of a patient's body part, comprising at least one first body part section (21) that is adjacent to at least one articulated second body part section (22), such that the first body part section (21) can be moved by means of the articulated second body part section (22), wherein the method comprises the following steps performed by a digital processing device (12):
providing digital, camera-based image data that contains the part of the patient's body to be digitally modelled from different imaging directions,
identifying the at least one first body part section (21) from the digital image data provided and creating a first 3D body part section model (31b, 32b) for the identified first body part section (21) based on the digital image data provided and depth information provided on the identified first body part section (21),
identifying the at least one articulated second body part section (22) from the digital image data provided, and
creating the digital 3D model of the body part to be modelled depending on the first 3D body part section model (31b, 32b) created and the identified articulated second body part section (22).

2. The method according to claim 1, characterized in that a second 3D body part section model (33b) for the identified articulated second body part section (22) is created based on the digital image data provided and depth information provided on the identified articulated second body part section (22), wherein the digital 3D model of the body part to be modelled is created depending on the first 3D body part section model (31b, 32b) created and the second 3D body part section model (33b) created.

3. The method according to claim 1 or 2, characterized in that at least two body part sections (21, 22) are further identified as a function of a relationship to one another.

4. The method according to one of the preceding claims, characterized in that the articulated second body part section (22) is identified as a function of a range of motion of the joint or that the first body part section (21) is identified as a function of a range of motion of a joint adjacent to the first body part section (21) or of the identified articulated second body part section (22).

5. The method according to one of the preceding claims, characterized in that the body part sections (21, 22) of the body part to be modelled are captured in a joint scanning procedure, wherein the at least one body part section model (31b, 32b, 33b) is created separately.

6. The method according to one of the preceding claims, characterized in that a machine learning system is provided which has learned a correlation of digital image data as input data with the respective identification of the corresponding body part section (21, 22) as output data, wherein the image data provided is entered into the machine learning system as input data for the purpose of identification and an identification of the corresponding body part section (21, 22) is then obtained as output data.

7. The method according to claim 6, characterized in that the machine learning system is an artificial neuronal network.

8. The method according to one of the preceding claims, characterized in that the digital, camera-based image data are continuously supplied to the digital processing device (12).

9. The method according to claim 8, characterized in that the 3D model of the body part is continuously created on the basis of the continuously provided image data.

10. The method according to one of the preceding claims, characterized in that, by means of a mobile image sensor, the digital, camera-based image data of the patient's body part to be modelled is recorded and supplied to the digital data processing device (12) in order to create the 3D body part model.

11. The method according to claim 10, characterized in that the mobile image sensor is a hand-operated image sensor.

12. A computer program with program coding means configured to carry out the method according to one of the preceding claims when the computer program is run on a data processing system.

13. A method for adjusting or producing an orthopedic device (100) of a patient fitted with the orthopedic device (100), comprising the steps:
creating a 3D body part model of the body part to be fitted with the orthopedic device (100) in accordance with the method according to one of the claims 1 to 11, and
adjusting or producing the orthopedic device (100) using the 3D body part model.
